(19) United States
(12) Patent Application Publication
(10) Pub. No.: US 2007/0048857 A1
(43) Pub. Date: Mar. 1, 2007

(54) Automatic macroinjection apparatus and cell trapping plate

(75) Inventors: Akio Ito, Kawasaki (JP); Akihiko Yabuki, Kawasaki (JP); Satoru Sakai, Kawasaki (JP); Moritoshi Ando, Kawasaki (JP); Sachihiro Youoku, Kawasaki (JP)

Correspondence Address: STAAS & HALSEY LLP, SUITE 700, 1201 NEW YORK AVENUE, N.W., WASHINGTON, DC, 20005, US

(73) Assignee: FUJITSU LIMITED, Kawasaki (JP)

(21) Appl. No.: 11/288,280

(22) Filed: Nov. 29, 2005

(30) Foreign Application Priority Data
Aug. 5, 2005 (JP) 2005-228445

(51) Int. Cl. C12M 1/00

(52) U.S. Cl. 435/283.1

(57) ABSTRACT

A cell trapping plate traps a cell by applying a negative pressure suction through a trapping hole provided therein. A capillary needle is stuck into the trapped cell to inject an injectant. The cell trapping plate includes trapping holes arranged at irregular intervals in directions of two coordinate axes in a two-dimensional orthogonal coordinate system.

### BACKGROUND OF THE INVENTION

1. Field of the Invention

The present invention relates to an automatic microinjection apparatus and a cell trapping plate used to inject an injectant into a cell, and more particularly, to a cell trapping plate with improved resistance to pressure break and an automatic microinjection apparatus using the cell trapping plate.

2. Description of the Related Art

In the field of life science and the like, it is quite common to use an automatic microinjection apparatus when a biological molecule such as a gene, an antibody, and a protein, and a compound (hereinafter, these are generically called “injectant”) are injected into a cell.

The automatic microinjection apparatus automates an operation of retaining a cell and an operation of sticking a fine hollow glass needle called a “capillary needle” into the cell and injecting the injectant filled in the capillary needle into the cell, so that the injectant can be injected into a large number of cells at high speed.

Techniques for sucking cells in trapping holes by negative pressure suction using a cell trapping plate provided with micro through holes from the back thereof, and trapping the cells are disclosed, for example, in Japanese Patent No. 2662215 in which a cell trapping plate having a structure such that concave portions in which individual cells are completely accommodated are formed therein and through holes are made in each bottom of the concave portions is disclosed, in Japanese Patent No. 2624719 in which a cell trapping plate having only simple through holes is disclosed, and in U.S. Pat. No. 5,262,128 and Japanese Patent No. 3035608 in which cell trapping plates each having trapping holes of which opening edge is funnel-shaped are disclosed.

However, the cell trapping plate used in the automatic microinjection apparatus may be broken by pressure. The trapping holes used for cell retention are extremely fine, and in order to make such fine through holes, the periphery of the trapping hole is in thin film form with a thickness of about 10 μm.

In the automatic microinjection apparatus, prior to sucking cells to the cell trapping plate and trapping the cells therein, the periphery of the cell trapping plate needs to be filled with a buffer solution such as phosphate-buffered saline. However, during this process, a large amount of pressure is applied to the cell trapping plate, and the thin film form may be broken by the pressure.

If the pressure to be applied is reduced in order to avoid pressure break, then the periphery of the cell trapping plate is not fully filled with the buffer solution, which does not allow a capillary needle to be precisely guided to the cell and to stick the capillary needle into it.

### SUMMARY OF THE INVENTION

It is an object of the present invention to at least solve the problems in the conventional technology.

An automatic microinjection apparatus according to one aspect of the present invention includes a cell trapping plate that traps a cell by applying negative pressure suction through a trapping hole provided therein, and a capillary needle that is stuck into the trapped cell to inject an injectant. The cell trapping plate includes trapping holes arranged at irregular intervals in directions of two coordinate axes in a two-dimensional orthogonal coordinate system.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram for explaining an injection method using an automatic microinjection apparatus;

FIG. 2 is an example of an image observed by an inverted optical system;

FIG. 3 is an example of an arrangement of trapping holes according to an embodiment of the present invention;

FIG. 4 is a schematic diagram of a dish unit with a buffer solution fed;

FIG. 5 is a schematic diagram for explaining surface tension on the interfaces created at the trapping holes on the cell trapping plate;

FIG. 6 is a schematic diagram for explaining a droplet produced in the trapping hole of the cell trapping plate;

FIG. 7 is a schematic diagram for explaining a flux produced in the trapping hole of the cell trapping plate;

FIG. 8 is a schematic diagram for explaining deflection of the membrane portion by negative pressure suction;

FIG. 9 is an example of an average pitch of the trapping holes in the arrangement of the trapping holes according to the present embodiment;

FIG. 10 is a flowchart of a processing procedure for setting the arrangement of the trapping holes according to the present embodiment;

FIG. 11 is a schematic diagram of the automatic microinjection apparatus according to the present embodiment;

FIG. 12 is a perspective view of the automatic microinjection apparatus according to the present embodiment;

FIG. 13 is a schematic diagram for explaining a process procedure of the automatic microinjection apparatus according to the present embodiment;

FIG. 14 is an example of a sequence of injection by the automatic microinjection apparatus according to the present embodiment;

FIG. 15 is an example of an arrangement of the trapping holes in the shape of a fan;

FIG. 16 is an example of an arrangement of the trapping holes in the shape of a concentric circle; and

FIG. 17 is an example of an arrangement of trapping holes according to a conventional technology.

### DETAILED DESCRIPTION OF THE PREFERRED EMBODIMENTS

Exemplary embodiments of the present invention are explained in detail below with reference to the accompanying drawings.

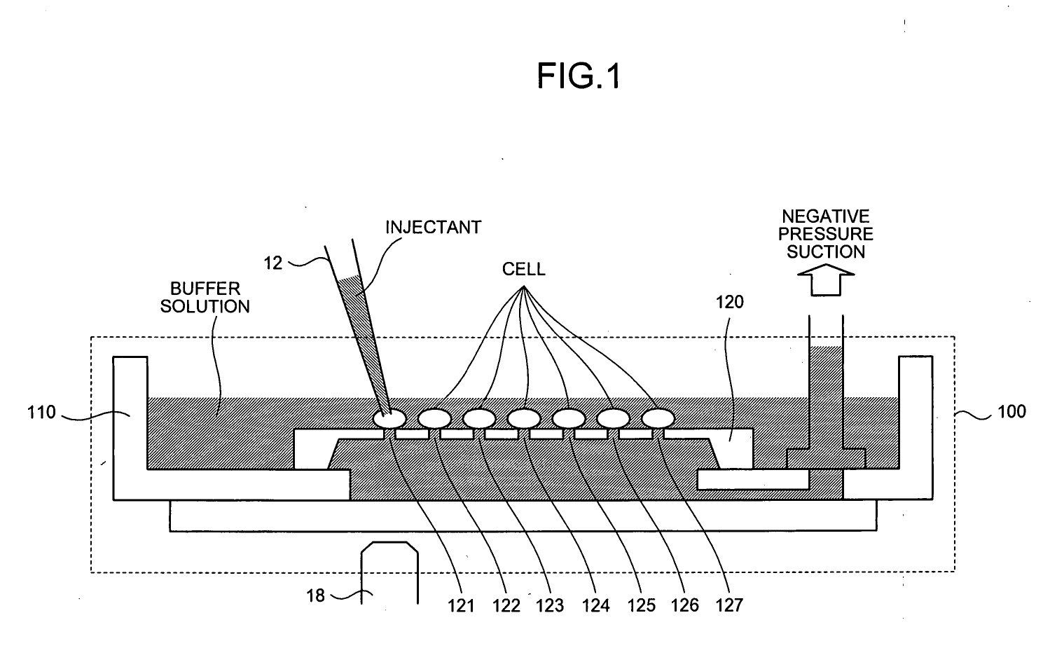

FIG. 1 is a schematic diagram for explaining an injection method using an automatic microinjection apparatus.

In a dish unit 100 used in the injection method, a cell trapping plate 120 is placed on a Petri dish 110 having a suction channel, and the dish unit 100 is filled with a buffer solution such as phosphate-buffered saline.

The cell trapping plate 120 has trapping holes 121 to 127 which are micro through holes, and trap cells, fed to the surface of the cell trapping plate 120, in the trapping holes 121 to 127, under negative pressure suction from below through the suction channel. In FIG. 1, there are shown seven trapping holes on the cell trapping plate 120 for simplicity of the drawing, but in actual cases, there is an extremely large number of trapping holes, as explained later.

In the automatic microinjection apparatus, a trapping hole is observed by an inverted optical system 18 from the back of the dish unit 100, and a capillary needle 12 filled with the injectant is guided to the trapping hole under observation. The capillary needle 12 is stuck into the cell trapped and the injectant is injected.

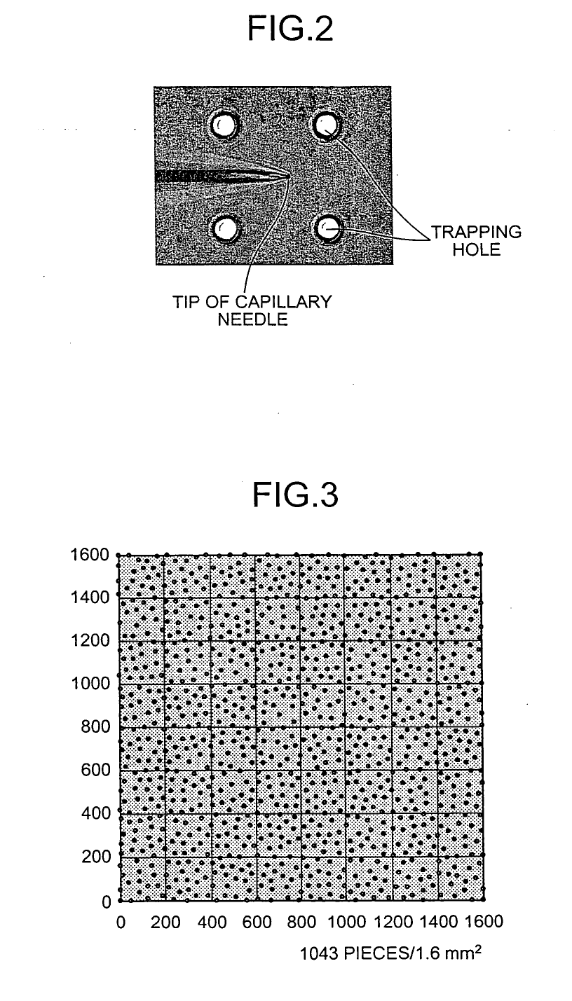

FIG. 2 is an example of an image observed by an inverted optical system. The image allows the automatic microinjection apparatus to observe a 3-dimensional tip position of the capillary needle 12 and a 3-dimensional position of the trapping hole at submicron accuracy, and to accurately adjust these positions.

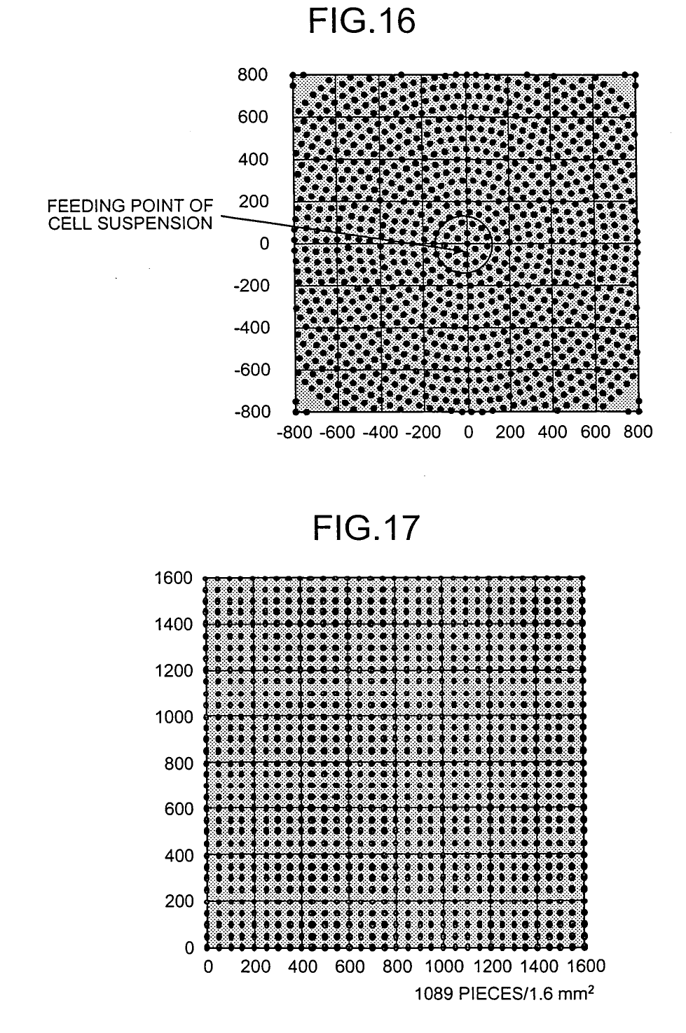

FIG. 17 is an example of an arrangement of trapping holes according to a conventional technology. In this example, 1089 pieces of trapping holes are provided in an area of 1.6 mm2. These trapping holes are arranged in a square lattice form. The reason that the trapping holes are arranged in the square lattice form is because the capillary needle 12 is easily guided.

FIG. 3 is an example of an arrangement of trapping holes according to the present embodiment. In this example, 1043 pieces of trapping holes are provided in an area of 1.6 mm2. These trapping holes are randomly arranged. The reason that the trapping holes are randomly arranged is because resistance to pressure break is improved while almost the same number of trapping holes as that in the conventional arrangement is arranged in the same area.

The cell trapping plate 120 undergoes the maximum pressure when a pre-sucking operation is performed in such a manner that the buffer solution is fed onto the cell trapping plate 120 and the cells are started to be sucked by the negative air pressure applied from the back of the cell trapping plate 120.

As shown in FIG. 1, it is necessary, for injecting the injectant, to observe the trapping holes by the inverted optical system 18 from the back of the dish unit 100. However, the features of an objective lens of the inverted optical system 18 are adjusted so as to accurately observe the cells trapped in the buffer solution. Therefore, the observation with high resolution can be performed only by filling a space in the back of the cell trapping plate 120 with the buffer solution.

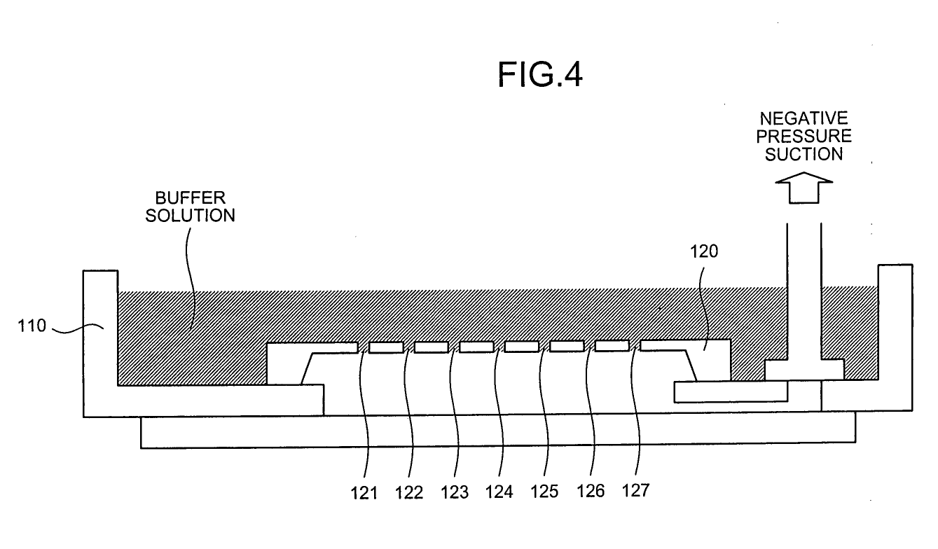

FIG. 4 is a schematic diagram of a dish unit with a buffer solution fed. As shown in the figure, only by feeding the buffer solution onto the cell trapping plate 120, interfaces between air and the liquid are created at the respective trapping holes, and strong surface tensions are acted to the interfaces, thereby the space is made. It is therefore necessary to apply suction by negative air pressure from the back of the cell trapping plate 120 and to fill the space with the buffer solution. This is the pre-sucking operation.

A target into which the injectant is injected by the automatic microinjection apparatus is in many cases somatic cells of human beings, and a diameter of an ordinary somatic cell is about 10 to 20 μm in suspension. The optimal diameter of the trapping hole to suck and trap the cell of this size is ⅓ to ⅕ of the cell diameter, i.e. about 2 μm to 4 μm.

If the diameter of the trapping hole is too large, the cell is sucked into the trapping hole and cannot be retained. If it is too small, sufficient trapping force is not provided, and hence, the cell moves while the capillary needle is being inserted into the cell, and injection cannot successfully be carried out.

It is an optimal method at present that a silicon substrate is used for the cell trapping plate and is treated by a semiconductor manufacturing process when a large number of through holes with a diameter of several micrometers is to be formed. Further, when the through holes are formed by using the semiconductor manufacturing process, the thickness of a plate at the through hole portion is about 10 μm at most from restriction by its aspect ratio.

Therefore, the back of the cell trapping plate 120 has to be largely scooped out, and the area where the trapping holes are arranged has to be a membrane (thin film) structure. If high pressure is applied to the membrane portion during the pre-sucking operation, the membrane portion is deflected as shown in FIG. 8 and may be broken in some cases.

FIG. 5 is a schematic diagram for explaining surface tension on the interfaces created at the trapping holes on the cell trapping plate 120. If the buffer solution is fed from the upper side of the cell trapping plate 120, then the buffer solution remains at the lower edge of the trapping hole by the surface tension.

An upward force at this time is obtained by
F_{UP}=2πrₕT sinθ (1)
where T is surface tension of liquid (for water: 0.072 N/m), and θ is a contact angle between the surface of the plate and liquid (for silicon and water: 30°).

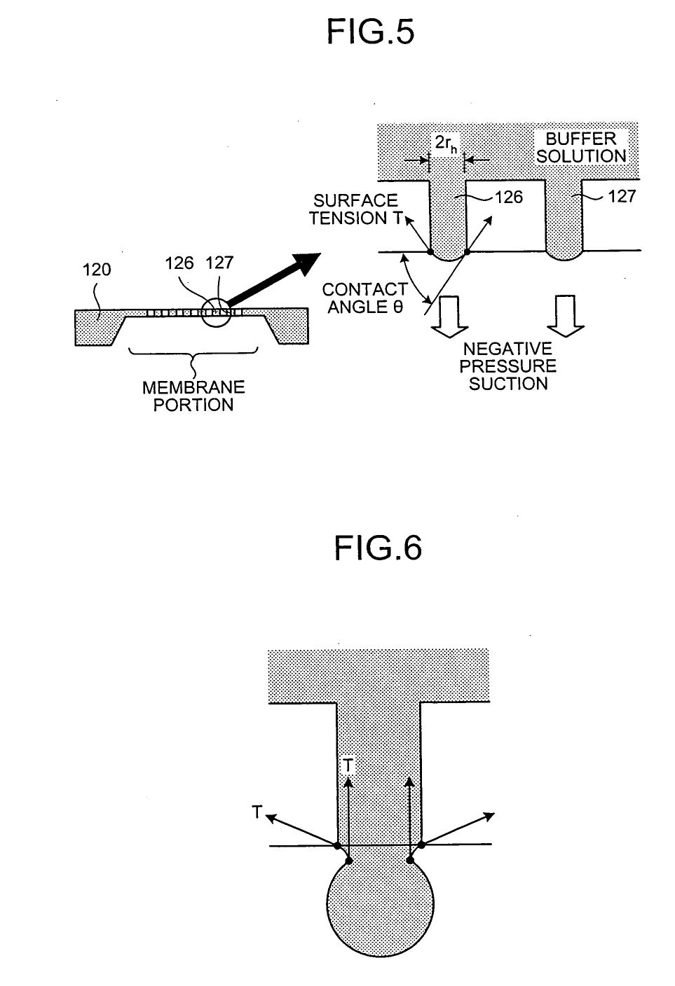

On the other hand, a downward force by suction pressure P is expressed as
F_{DOWN}=πrₕ²P (2)
Therefore, if the pressure P explained below satisfies a condition (F_{DOWN}>F_{UP}) such that the downward force becomes greater than the upward force, then a droplet grows as shown in FIG. 6.

$$P > \frac{2 T sin θ}{{r}_{h}} \qquad ( 3 )$$

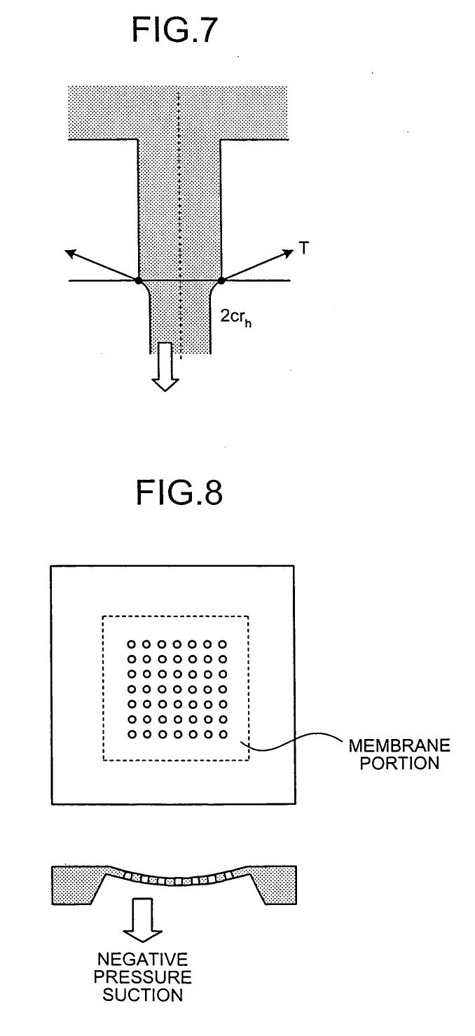

The size of the droplet increases as time elapses, and the droplet drops when the weight of the droplet exceeds the tension at the neck of the droplet. Alternatively, when the suction pressure P is sufficiently large, the lower-part interface is immediately broken to become a flux of 2 crₕ (c<1) in diameter as shown in FIG. 7, where c is a constant which is called a flow rate coefficient and is smaller than 1.

Here, it is understood that Equation (3) indicates the pressure required for the pre-sucking operation, and that a larger pressure is required if the inner diameter is smaller. For example, when the trapping hole is 3 μm in diameter, 48 kPa is required as a previous suction pressure.

Since the pressure is applied to the membrane portion with the thickness of 10 μm, for example, the central portion of a silicon membrane is deflected even by several 10 μm, and the membrane portion may be broken in some cases.

Strength against breakage of the membrane portion largely relates to not only mechanical properties of a material but also to the presence of the large number of through holes provided in the membrane. Particularly, as shown in the conventional manner, in the cell trapping plate on which the trapping holes are regularly arranged and evenly spaced therebetween in a square lattice form, through hole arrays are arranged on vertical and horizontal lines, respectively. Therefore, stress concentration points at the respective trapping holes due to distortion of the membrane are linearly aligned to become a band shape, and the membrane is prone to be broken along the band-shaped portion as a starting point.

Since the silicon membrane in particular is a single crystal substrate, the array of the trapping holes perfectly coincides with a crystal axis that is easy to cleave, and hence, the strength against breakage is largely reduced.

A deflection w of a rectangular plate undergoing a distributed load P satisfies a differential equation expressed as (Reference: “Strength of Current Materials”, Shibuya, et al., Asakura Shoten, p 211, 1986)

$$\frac{{\partial }^{4} w}{\partial {x}^{4}} + 2 ( \frac{{\partial }^{4} w}{\partial {x}^{2} \partial {y}^{2}} ) + \frac{{\partial }^{4} w}{\partial {y}^{4}} = \frac{P}{D} \qquad ( 4 )$$

where D is the flexural rigidity of a plate with a thickness t, and it is obtained by

$$D = \frac{{Et}^{3}}{12 ( 1 - {v}^{2} )} \qquad ( 5 )$$

where E is Young's modulus and v is Poisson's ratio.

The membrane portion of the cell trapping plate can be regarded as a square plate of which four sides undergoing a evenly-distributed load are fixed (a length of one side: L, thickness: t). In this case, if Equation (4) is solved, then it is understood that the maximum deflection wₘₐₓ is produced at a center position of the membrane portion, and that the maximum stress σₘₐₓ is produced at the top surface and back side of the center position. Both of these are approximately given by Equations (6) and (7), respectively, where, when v=0.3, μ₁ =0.00126 and μ₂=0.0513.

$$\begin{matrix}{w}_{max} = \frac{12 {μ}_{1} ( 1 - {v}^{2} ) {PL}^{4}}{{Et}^{3}} & ( 6 ) \\ {σ}_{max} = \frac{6 {μ}_{2} {PL}^{2}}{{t}^{2}} & ( 7 )\end{matrix}$$

In actual cases, since the stress is concentrated around the edge of the trapping hole, the maximum stress applied to the plate is definitely larger than the value of Equation (7), but it cannot be calculated so easily. Therefore, the maximum stress is evaluated by using Equation (8) in which the value of the Equation (7) is multiplied by a stress concentration coefficient α in the deflection of a band plate having circular holes.

$${σ}_{max} = \frac{6 α {μ}_{2} {PL}^{2}}{{t}^{2}} \qquad ( 8 )$$

When a hole diameter/band width (pitch) is 12/50=0.24, the stress concentration coefficient α at this time becomes 1.44 (Reference: “Mechanical Engineering Handbook”, A49-98, 2001)

Assume that the membrane portion is a square, L=1.7 mm on a side, and its thickness is 10 μm. The distributed load when the maximum stress σₘₐₓ exceeds the breaking stress of silicon, i.e. a breaking pressure Pₘₐₓ becomes −40 kPa, and this indicates that the pressure required for the previous suction slightly exceeds the breaking pressure.

The maximum amount of deflection under this pressure reaches even 36 μm. The deflection produces a large magnitude of stress near the trapping hole. In the conventional membrane on which the trapping holes are regularly arranged in the square lattice form, the stress concentration points are aligned in a row, and this causes the strength against breakage of the membrane to be largely reduced.

In the calculation, the silicon surface of the plate is assumed to have values as follows: Young's modulus=130.8 Gpa, Poisson's ratio v=0.28, and breaking stress σ=500 MPa.

Since the number of cells that can be treated at a time by a piece of cell trapping plate is decided by the number of trapping holes on the cell trapping plate, at least 1,000, possibly 10,000 through holes are required. If there are 10,000 trapping holes, the membrane portion having a further larger area is required, and the risk of its breakage further increases.

Since the cell trapping plate 120 according to the present invention has the trapping holes which are arranged at irregular intervals in respective coordinate axis directions in a two-dimensional orthogonal coordinate system, the points where the stress is concentrated are not formed linearly in a band shape. Therefore, the strength against breakage is largely improved. Particularly, when the cell trapping plate is the silicon substrate and the trapping holes are arranged 2-dimensional randomly, the arrangement allows the array of the trapping holes formed along the silicon crystal axis to be largely reduced, and hence, the strength against breakage is largely improved.

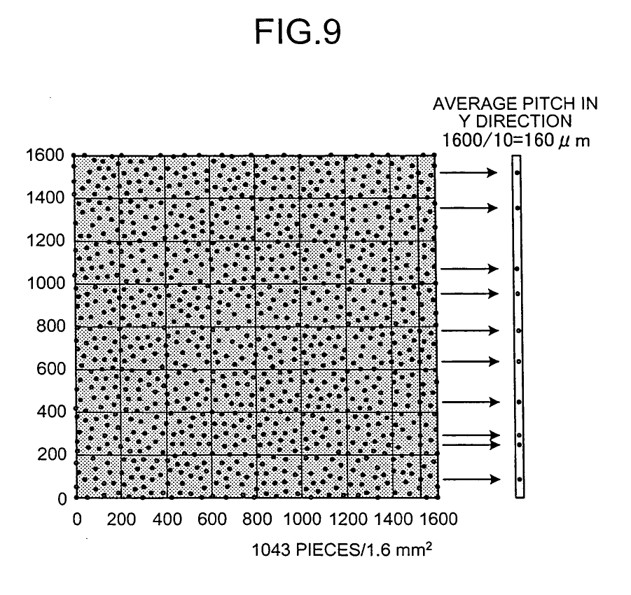

When the trapping holes are randomly arranged, an average pitch of the trapping holes aligned in a line becomes much longer as compared with that in the lattice-shaped arrangement. The pitch of the trapping holes in the conventional example of the arrangement shown in FIG. 17 is 50 μm. On the other hand, in the case of random arrangement as shown in FIG. 9, as a result of determining an average pitch of trapping holes on lines along some coordinate axes, it is found that the average pitch ranges from 90 to 160 μm, which is longer by 1.8 to 3.2 times than that of the conventional case.

Accordingly, in the random arrangement, the influence of presence of the through holes is significantly decreased. In the arrangement in the square lattice form, the suction pressure required for the pre-sucking operation is almost the same level as the strength against breakage, while the random arrangement allows reinforcement of the strength against breakage to such an extent that there is no need to worry about the strength against breakage during the pre-sucking operation.

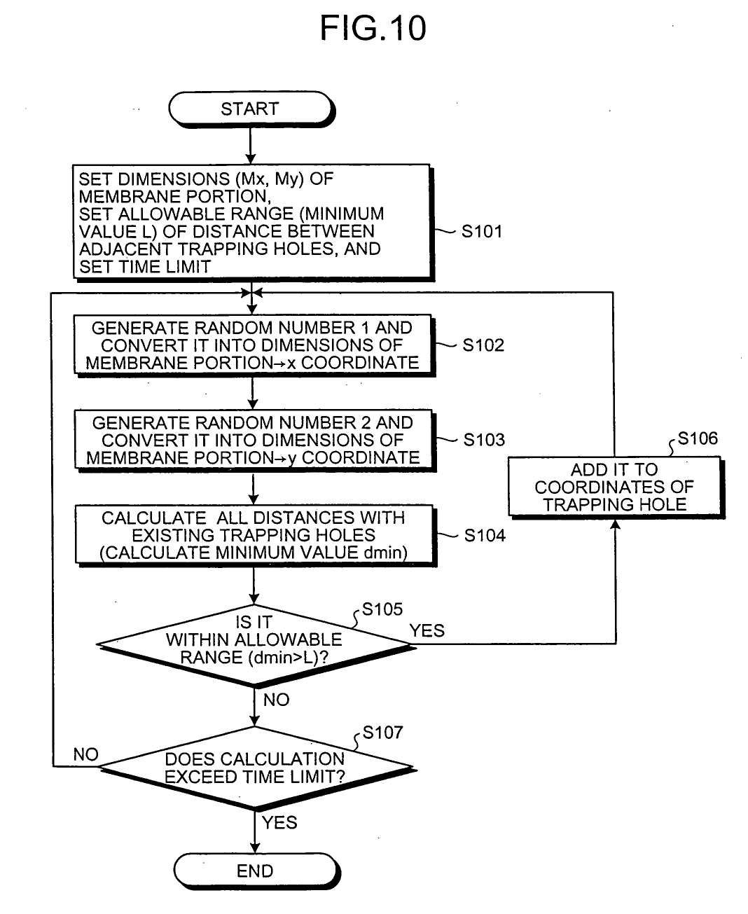

FIG. 10 is a flowchart of a processing procedure for setting the arrangement of the trapping holes according to the present embodiment.

As shown in FIG. 10, settings are performed on dimensions Mx of the membrane portion in the X-axis direction, dimensions My of the membrane portion in the Y-axis direction, an allowable minimum value L of a distance between adjacent trapping holes, and a time limit (step S101). If the trapping holes are too close to each other, the capillary needle may erroneously catch in a neighboring cell upon injection. Therefore, the allowable minimum value L is appropriately 2 to 3 times of the diameter of a target cell.

A first random number is generated, this number is multiplied by Mx to be converted to the dimensions of the membrane portion, and a value converted is set as an x coordinate value of a temporary trapping hole (step S102). Likewise, a second random number is generated, this number is multiplied by My to be converted to the dimensions of the membrane portion, and a value converted is set as a y coordinate value of a temporary trapping hole (step S103).

Then, all the distances each between one of existing trapping holes and a temporary trapping hole are obtained, and the minimum value of the distances is set as dmin (step S104). If dmin is greater than the allowable minimum value L (step S105, Yes), then the temporary trapping hole is added as a proper trapping hole (step S106), and process returns to step S102, where the coordinates of the next temporary trapping hole are obtained.

If dmin is smaller than the allowable minimum value L (step S105, No), then it is checked whether an elapsed time from the start of processing exceeds the time limit. If the elapsed time does not exceed the time limit (step S107, No), then process returns to step S102, where the coordinates of the next temporary trapping hole is obtained, while if the elapsed time exceeds the time limit (step S107, Yes), then the process is ended.

The arrangement of the trapping holes acquired in the above manner is used when the cell trapping plate 120 is manufactured and when the automatic injection is operated.

Since there is sometimes a case where the sufficient number of trapping holes is not ensured in the process procedure, it is preferable to repeat the process some times and select an optimal arrangement as a result. The arrangement of the trapping holes may be set in another process procedure.

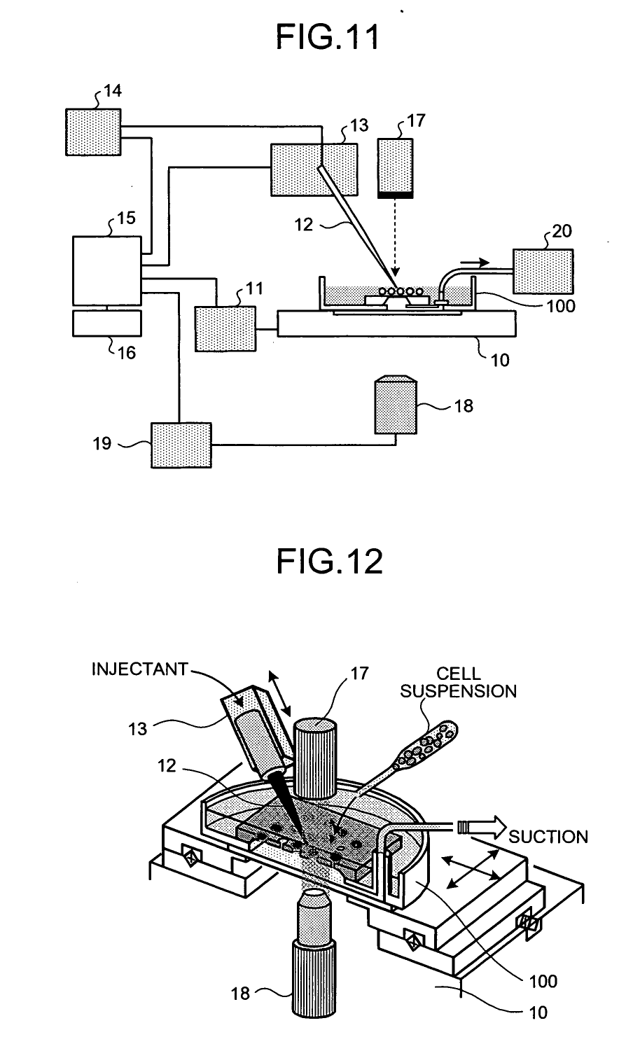

The configuration of the automatic microinjection apparatus according to the present embodiment is explained below. FIG. 11 is a schematic diagram of the automatic microinjection apparatus according to the present embodiment.

As shown in FIG. 11, the automatic microinjection apparatus according to the present embodiment includes an XY stage 10, an XY-stage control unit 11, the capillary needle 12, a manipulator 13, a dispense mechanism 14, a computer 15, a trapping-hole-coordinate storing unit 16, a illuminator 17, the inverted optical system 18, a camera 19, and an air-pressure control unit 20.

The XY stage 10 is a base on which the dish unit 100 is mounted, and can move in the X-axis direction and Y-axis direction under the control of the XY-stage control unit 11. The XY-stage control unit 11 is a control unit that controls the movement of the XY stage 10 according to an instruction of the computer 15.

The capillary needle 12 is a fine hollow glass tube for injecting an injectant, and is held by the manipulator 13. The manipulator 13 is a device that holds the capillary needle 12 and controls the operation of pushing it out/pushing it back. The dispense mechanism 14 is a mechanism for dispensing the injectant filled in the capillary needle 12 from the tip thereof.

The computer 15 is a controller that controls the whole of the automatic microinjection apparatus, and executes various automatic processes. For example, in the injection process, the controller acquires coordinate information for each trapping hole in the cell trapping plate 120 placed on the dish unit 100, from the trapping-hole-coordinate storing unit 16, and moves the XY stage 10 based on the information. Then, the controller sequentially and automatically executes the processes of observing an image to be captured by the inverted optical system 18, performing accurate positioning of a trapping hole, and introducing the injectant into the capillary needle 12.

The trapping-hole-coordinate storing unit 16 is a unit that stores coordinate information for each trapping hole of the cell trapping plate 120. In the conventional arrangement of the trapping holes in the square lattice form, by storing only the pitches of the trapping holes and the number of trapping holes in rows and columns, respective positions of the trapping holes can be obtained by simple computations. However, in the automatic microinjection apparatus according to the present embodiment, because the trapping holes are randomly arranged, the coordinate information for the trapping holes needs to be stored.

The trapping-hole-coordinate storing unit 16 may previously store the coordinate information for the trapping holes of all types of cell trapping plates, or may read out the coordinate information stored in a storage medium such as a memory card when the automatic microinjection operation is started, and hold it. Alternatively, the trapping-hole-coordinate storing unit 16 may download the coordinate information through the network and hold it.

The illuminator 17 radiates light from the upper side of the dish unit 100 toward the periphery of the trapping holes in order to make clear an image to be captured by the inverted optical system 18. The inverted optical system 18 is an optical unit that captures an image around the trapping hole from the lower side of the dish unit 100. The camera 19 is a device that converts the image captured by the inverted optical system 18 to electronic data so that the computer 15 can recognize it.

The air-pressure control unit 20 is a controller that controls generation of negative pressure required for the pre-sucking operation and the cell trapping operation.

FIG. 12 is a perspective view of the periphery of the XY stage 10 of the automatic microinjection apparatus according to the present embodiment. As shown in the figure, target cells to be injected are fed into the dish unit 100 as a cell suspension from the upper side thereof, and are trapped in trapping holes by the cell trapping operation.

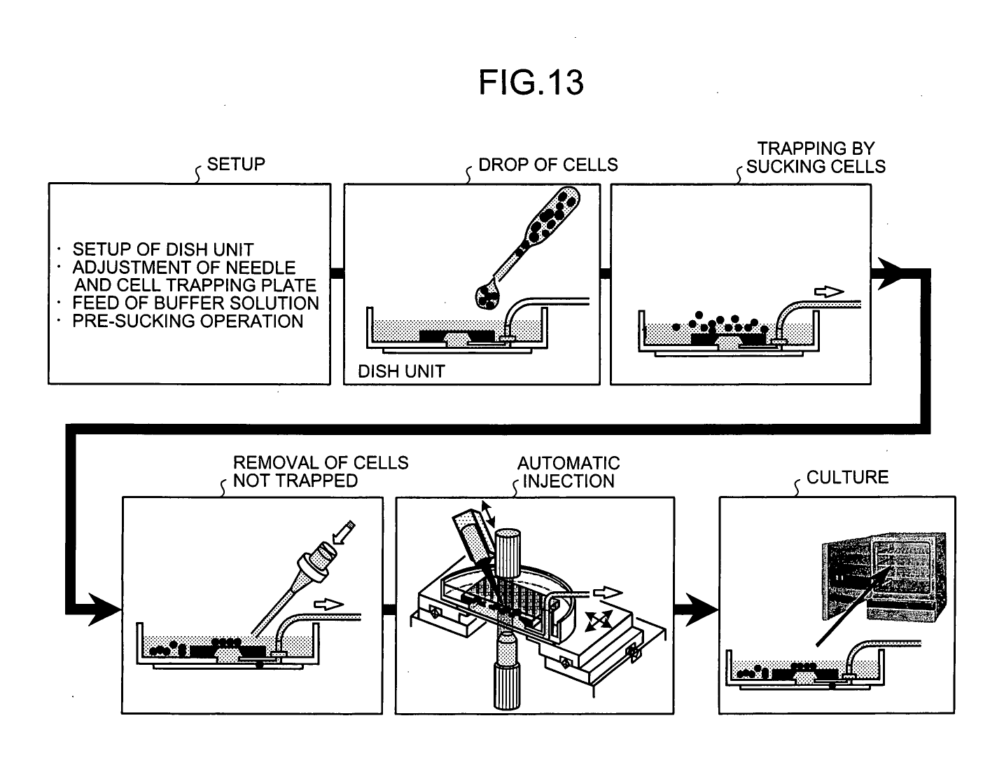

FIG. 13 is a schematic diagram for explaining a process procedure of the automatic microinjection apparatus according to the present embodiment.

A setup including processes as follows is performed, which includes adjustment of the positions of the cell trapping plate 120 and the capillary needle 12, feed of the buffer solution, and a pre-sucking operation. Then, a cell suspension is dropped by a syringe, an appropriate negative pressure (−several 100 Pa) is applied from the back of the cell trapping plate, and cells floating in the suspension are trapped in the trapping holes to be retained so as not to move. Unnecessary cells remaining without being trapped are washed out with the buffer solution, to be removed, and automatic injection is sequentially performed into the cells trapped.

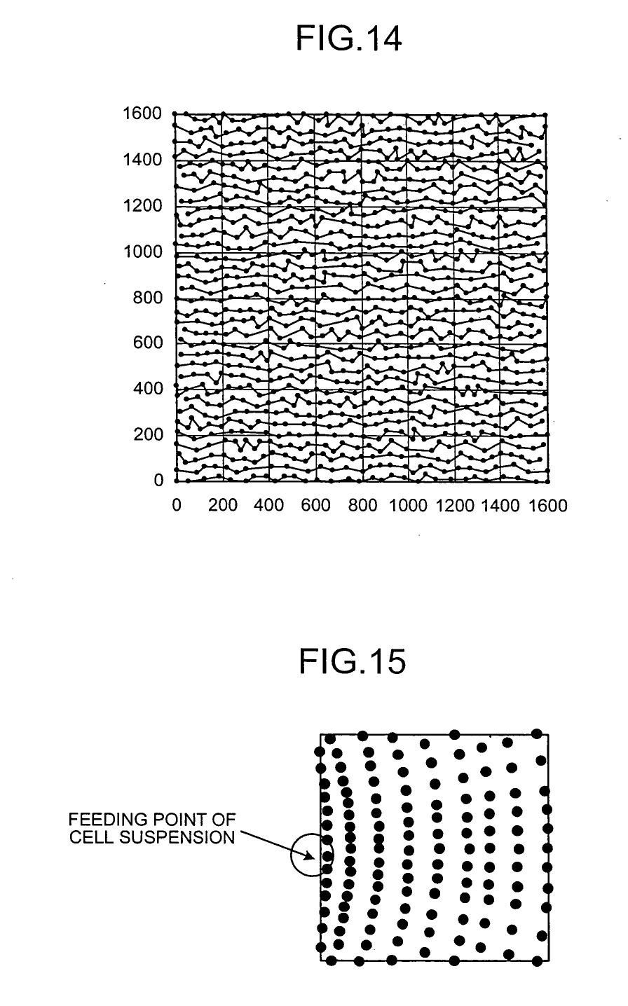

FIG. 14 is an example of a sequence of injection by the automatic microinjection apparatus according to the present embodiment. As shown in the figure, coordinate data for trapping holes is sorted for each area obtained by dividing positions of trapping holes into band-shaped areas in the X-axis direction, and movement of the XY stage 10 can be thereby suppressed to the minimum.

After the injection operation to all the cells trapped is complete, the whole dish unit 100 is sent to the next treatment process such as culture and observation.

According to the present embodiment, since the trapping holes on the cell trapping plate are randomly arranged, the average pitch of the holes aligned along the line is increased as compared with that of the lattice-shaped arrangement, which allows improvement of the resistance to pressure break.

Accordingly, the pre-sucking operation is made easier, which leads to improved reliability of the cell trapping, and hence, a larger membrane area can be used. Therefore, the number of trapping holes can be thereby increased, and much more cells can be treated at a time. Particularly, when the cell trapping plate is made from a silicon substrate, an average interval between trapping holes aligned along the crystal axis which is easy to cleave is made longer, and hence, the effect in improvement of strength against breakage becomes large.

Since the random arrangement of the trapping holes is similar to how cells exist in nature, such an effect that a favorable influence is given to existence of the cells can also be expected.

Although the example of randomly arranging the trapping holes to improve the strength against breakage of the cell trapping plate is explained in the present embodiment, the same effect can also be obtained if the trapping holes are arranged in the shape of a fan or a concentric circle.

FIG. 15 is an example of an arrangement of the trapping holes in the shape of a fan. This figure shows an arrangement of the trapping holes in a fan shape on the cell trapping plate with a cell feeding point as a pivot. In the arrangement also, the trapping holes can be avoided from being aligned at regular intervals in lines orthogonal to each other. Therefore, this arrangement also has a certain effect in improvement of the strength against breakage of the cell trapping plate. Furthermore, in the present embodiment, since the trapping holes are arranged along a flow along which the cells having been fed are dispersing by themselves, this arrangement has an effect in improvement of a trapping rate of cells (the number of cells actually trapped/the number of trapping holes).

FIG. 16 is an example of an arrangement of the trapping holes in the shape of a concentric circle. Even if the trapping holes are arranged concentrically or spirally, the trapping holes are not aligned at regular intervals when viewed from the 2-dimensional coordinate axes orthogonal to each other, and hence, this arrangement has an effect in improvement of strength against breakage of the cell trapping plate. Moreover, the arrangement is provided along a natural flow of cells when the cells are fed from the center of the cell trapping plate. Therefore, this arrangement has an effect also in improvement of the trapping rate of cells.

Although the case where silicon is used as a material of the cell trapping plate is explained in the present embodiment, the strength against breakage can be improved even if plastic is used by arranging the trapping holes in the above manner. When the plate is made of plastic and the trapping holes are arranged at irregular intervals in the directions of two coordinate axes in the 2-dimensional orthogonal coordinate system, the cell trapping plate with high resistance to the pressure break can be obtained at low cost.

As described above, according to the present invention, the average pitch of the holes aligned in a line becomes much longer as compared with that in the lattice-shaped arrangement, which allows improvement of the resistance to pressure break.

Furthermore, according to the present invention, even if the trapping holes are arranged at irregular intervals in the directions of two coordinate axes in the 2-dimensional orthogonal coordinate system, the injection operation can be automatically controlled.

Moreover, according to the present invention, the cell trapping plate with high resistance to pressure break can be provided at low cost.

Furthermore, according to the present invention, the average pitch of the holes aligned in a line becomes longer as compared with that in the lattice-shaped arrangement, which allows improvement of the resistance to pressure break.

Although the invention has been described with respect to a specific embodiment for a complete and clear disclosure, the appended claims are not to be thus limited but are to be construed as embodying all modifications and alternative constructions that may occur to one skilled in the art that fairly fall within the basic teaching herein set forth.

What is claimed is:

1. An automatic microinjection apparatus comprising:
a cell trapping plate that traps a cell by applying a negative pressure suction through a trapping hole provided therein; and
a capillary needle that is stuck into the trapped cell to inject an injectant, wherein
the cell trapping plate includes trapping holes arranged at irregular intervals in directions of two coordinate axes in a two-dimensional orthogonal coordinate system.

2. The automatic microinjection apparatus according to claim 1, further comprising:
a storing unit that stores arrangement information of the trapping holes provided on the cell trapping plate; and
a control unit that guides the capillary needle to the trapping hole where the cell, to which the injectant is injected, is trapped, based on the arrangement information stored by the storing unit.

3. The automatic microinjection apparatus according to claim 1, wherein
the cell trapping plate is made of either one of silicon and plastic.

4. The automatic microinjection apparatus according to claim 1, wherein
the trapping holes are randomly arranged while keeping a predetermined distance or more from adjacent trapping holes.

5. The automatic microinjection apparatus according to claim 1, wherein
the trapping holes are arranged in a shape of a fan.

6. The automatic microinjection apparatus according to claim 1, wherein
the trapping holes are arranged in a concentric pattern.

7. The automatic microinjection apparatus according to claim 1, wherein
the trapping holes are arranged in a spiral manner.

8. A cell trapping plate for trapping a cell in an automatic microinjection apparatus, the cell trapping plate comprising:
a plurality of trapping holes arranged at irregular intervals in directions of two coordinate axes in a two-dimensional orthogonal coordinate system.

9. The cell trapping plate according to claim 8, wherein
the cell trapping plate is made of either one of silicon and plastic.

10. The cell trapping plate according to claim 8, wherein
the trapping holes are randomly arranged while keeping a predetermined distance or more from adjacent trapping holes.

11. The cell trapping plate according to claim 8, wherein
the trapping holes are arranged in a shape of a fan.

12. The cell trapping plate according to claim 8, wherein
the trapping holes are arranged in a concentric pattern.

13. The cell trapping plate according to claim 8, wherein
the trapping holes are arranged in a spiral manner.
